(19) United States
(12) Patent Application Publication
(10) Pub. No.: US 2003/0086988 A1
(43) Pub. Date: May 8, 2003

(54) Method for manufacturing nutricious supplement food and nutricious supplement food

(76) Inventor: Teruaki Iwasaki, Hokkaido (JP)

Correspondence Address: MCKEE, VOORHEES & SEASE, P.L.C., 801 GRAND AVENUE, SUITE 3200, DES MOINES, IA, 50309-2721, US

(21) Appl. No.: 10/187,524

(22) Filed: Jul. 2, 2002

Related U.S. Application Data
(63) Continuation-in-part of application No. 09/740,520, filed on Dec. 19, 2000, now abandoned.

(51) Int. Cl. A61K 35/78

(52) U.S. Cl. 424/750; 426/18

(57) ABSTRACT

This invention relates to a nutritious supplemental food containing excellent nutritive substances that upon performing a continuous taking of a specified amount of food, it may provide restriction of active oxygen in the human body, netrition lesion of blood flow and a superior effect for activation of metabolism. A mixture of embryo and seed coat of unpolished rice as a major material heated with steam, and species Koji fungi are mixed to cultivate Koji on the culture bed, thereafter Koji has been holded at a specified temperature and time. Then, species Koji fungi contained in Koji is killed and a dried species Koji fungi cultivation mixture with a little moisture content without loosing any capability of catalysis of each enzyme group contained species Koji fungi is obtained. A method for manufacturing thereof is characterised in that said dried species Koji fungi cultivation mixture applied as a major substance is mixed with a sub-material which does not cultivate any species Koji fungi.

### BACKGROUND OF THE INVENTION

1. Field of the Invention

This invention relates to food having a superior activated action for metabolism and further showing a superior depression against occurrence of diabetes in view of its experiment; a method for manufacturing food providing an effect of improvement of a physical constitution upon taking a specified amount of food in a continuous manner for a certain period of time and suitable for keeping healthy condition; and nutritious supplemental food.

More particularly, this invention relates to a method for manufacturing nutritious supplemental food and nutritious supplemental food in which mixtures of embryo and seed coat of unpolished rice are heated with steam as a major material, the steamed mixtures are further mixed with Aspergills oryzar strain group being species Koji fungi acting as aerobe, they are cultivated and ripened while both their temperature and humidity are being adjusted, thereafter the species Koji fungi in the species Koji fungi cultivation mixture is perished, the included enzyme group such as amylase, protease, lipase, etc. are changed into dried substance under a state in which the capability of catalysis is not lost, the dried species Koji fungi cultivation mixture is applied as major substance and sub-material supplementing nutritious elements is added as required; and the nutritious supplemental food.

2. Description of the Related Art

It is known that fat metabolism failure and carbohydrate metabolism failure in a human body and the like are one of derangements caused by active oxygen in a physical body. Either derangement caused by oxygen or derangement in cells such as various kinds of inflammation are generated at lipid of vivimembrane, enzyme protein, and DNA (deoxyribonucleic acid) and the like by each of nuclides of the active each of nuclides of the active oxygen (superoxide anionic radical: .0₂—, hydroxy radical: .OH). It is said that, β cells secreting insulin in pancreas, for example, may generate inflammation by .OH to reduce secreting of insulin and to increase blood sugar value.

In general, lipid in a physical body is changed into lipoprotein (complex body of lipid and protein) and present in blood. It is said that if metabolism failure occurs in this lipoprotein, cerebral infarct, arteriosclerosis, and ischemic malum cordis or the like occur.

In the case of diabetes, if this disease is left without applying any medical treatment, eyes and capillary blood vessels are weakened as its complication.

In the case of autoimmunization type I diabetes, β cells in pancreas secreting insulin are attacked by self B limph corpuscles, the number of cells is reduced and the cells are diminished.

In the case of type II diabetes, the number of insulin receptor is decreased by increasing fat caused by overeating or becoming fat, resulting in that this type II diabetes may become the same state as that caused by a lack of insulin. Although the β cells may produce insulin as the blood sugar value is increased, it may invite a functional fatigue to cause its syndrome to become chronic.

An example of a blood pressure depressant has already been disclosed in the gazette of Japanese Patent Publication No.Hei 8-40, wherein rice brans (such as rice embryo, defatted rice embryo, rice bran and defatted rice bran etc.), soy beans (defatted soy beans, soybean flour, soybean powder, soybean refuse and their hydrolysis substances etc.) are fermented at alkaline liquid medium with pro-alkaline bacillus subtilis (anaerobic bacteria) and then their extract is extracted.

As disorder in metabolism function concerning vegetative, a deterioration of carbohydrate metabolism failure is a typical one and this has a substantial meaning as an index for an aging (a degree of aging). As a rate of high-aged population is increased, an age where a high rate of decreasing in function of carbohydrate metabolism is assumed to be about age 65. A nutritious disorder caused by reduction in this carbohydrate metabolism may induce a lack of energy and become a major cause of disorder in healthy condition, resulting in that a series of syndrome are produced. These matters are applied as an important subject matter to be solved in the future of coming high-aged society.

It is an object of the present invention to provide a nutritious supplemental food capable of mainly increasing this carbohydrate metabolism function and fat metabolism function.

It is estimated that the number of patients suffering from auto immunization type I diabetes receiving a medical treatment such as application of insulin and the like exceeds 5 million people all over the world. Since synthetic insulin is different in regard to a composing ratio of amino acid from that of human being, it is said that its long term use reduces its effectiveness. In recent years, an increased number of fat children and children's diabetes become social concern.

In turn, it is known that fermentation of food increases an amount of enzyme in the food. An effect of nutrition of food having its rate of absorption of enzyme into a physical body of living thing has been noticed. However, in the case of cultivation of species Koji fungi in the food, contamination other than the purpose of cultivation must be avoided, resulting in that this cultivation has shown a technical important problem in view of control of food temperature at the culture bed in addition to control over temperature of and humidity of atmosphere in the culture bed.

A cubic structure of sugar chain in glycoprotein is complex and its physiological meaning is not yet analyzed well. It has already been known in medical field that the functions of glycoprotein are deeply concerned with intercell acknowledgment caused by difference in structure of sugar chain, motion of cells in the physical body and protein metabolism of the cells.

In particular, it is estimated that variation in sugar chain induced by active oxygen or virus or the like may disturb the inter-cell acknowledgment and produce disorder of receptor and this state may appear as either metabolism failure or immunodeficient.

### SUMMARY OF THE INVENTION

The present inventor has continued to study earnestly and completed the present invention of a method for manufacturing food containing a large amount of group of enzyme such as amylase, protease, lipase and the like generated by Koji fungi to embryo of unpolished rice in reference to the fact that when glycoprotein of the embryo, sugar chain and the group of enzyme not losing any capability of catalysis action generated by Koji fungi are received within the physical body, they may activate hormone system and the physiological activating substance and the activated metabolism may contribute to an improvement of various kinds of diseases.

In the present invention, such a technical means as described below has been employed to overcome the aforesaid problem. That is, the present invention may provide a nutritious supplemental food in which either a single embryo portion of unpolished rice, in particular, in grain or mixture of embryo and seed coat is heated with steam, a group of Aspergills oryzae strain of species Koji fungi acting as aerobe (hereinafter merely called as species Koji fungi), it is cultivated and ripened on the culture bed, thereafter the species Koji fungi is diminished, the group of enzyme is dried while the capability of catalysis action is not lost, the dried species Koji fungi cultivation mixture powder is applied as major material and then sub-material not cultivated with species Koji fungi.

Definition of each of the specified technical terms used in the specification is as follows.

The term, “major material” denotes embryo and seed coat of unpolished, which will be major substance cultivating Koji fungi.

The term, auxiliary material” denotes material heated with steam together with the major material, wherein the auxiliary material means unpolished rice and Ganoderma lucidum.

The term, “sub-material” denotes material that is not cultivated with species Koji fungi which will be applied with species Koji fungi cultivation mixture.

The term “mixed major material” denotes material that Koji fungi are mixed to major material.

The term, “species Koji fungi cultivation mixture” denotes mixed major material, with which species Koji fungi are cultivated.

The term “dried species Koji fungi cultivation mixture” denotes species Koji fungi cultivation mixture, in which the contained moisture is dried from 2.5 wt % to 3.6 wt % after Koji fungi are ripened.

“Species Koji fungi” means Aspergillus oryzae stain which is cultivated at the major material or auxiliary material.

Ripening means that species Koji fungi is cultivated at the major material or the auxiliary material, either the major material or the auxiliary material is sufficiently decomposed into low molecules with enzyme secreted by the species Koji fungi are fixed there.

More practical content of the present invention will be described as follows.

(1) There are provided a method for manufacturing a nutritious supplemental food comprising:

as its initial step, heating a mixture of embryo and seed coat of unpolished rice as a major material with steam and cultivating a mixed major material having said heated mixture mixed with Aspergills oryzae strain group of species Koji fungi for 32 hours to 36 hours on a culture bed with atmosphere of 31° C. to 37° C. and humidity of 90%RH to 100%RH while both temperature and humidity being adjusted in a stepwise manner;

as its guiding step, cultivating said mixed major material for a minimum period of 8 hours agitating the mixed major material in the culture bed in order to stop an increasing in a heat generating temperature of said mixed major material generated by cultivating the species Koji fungi and thereby dropping its temperature to a scope ranging from 32° C. to 41° C.;

as its growing step, keeping the atmosphere at 31° C. to 36° C., keeping a temperature of said mixed major material in the culture bed at 40° C. to 43° C., continuing cultivation for 6 hours, thereafter cultivating the mixed major material for 11 hours to 12 hours controlling a temperature of the mixed major material in the culture bed at 39° C. to 40° C. and ripening it;

as its stabilizing step, keeping the atmosphere at a temperature of 36° to 37° C. and a humidity of 90%RH to 100%RH, adjusting a temperature of said mixed major material in the culture bed to 39.5° C. to 40° C. and ripening it for 6 hours to attain a species Koji fungi cultivation mixture;

as a drying step, holding the ripened species Koji fungi cultivation mixture in the culture bed at 44° C. to 46° C. for 8 hours and 30 minutes to 9 hours and 30 minutes without controlling an increasing of heat generating temperature of it, thereafter killing the species Koji fungi contained in the species Koji fungi cultivation mixture on the culture bed with hot heat in which a temperature of said species Koji fungi cultivation mixture is controlled to 39° C. to 40° C., to attain a dried species Koji fungi cultivation mixture with moisture content of 2.5% to 3.6% without losing any capability of catalysis of each enzyme group contained in said species Koji fungi cultivation mixture with heat of said species Koji fungi cultivation mixture; and

under a combination of these steps, mixing said dried species Koji fungi cultivation mixture applied as a major substance with a sub-material which does not cultivate any species Koji fungi.

(2) There is provided a nutritious supplemental food having as its major substance the dried species Koji fungi cultivation substance powder in which the species Koji fungi Aspergills oryzae strain group is cultivated and ripened at the mixture of embryo and seed of raw unpolished rice composed of grain and the enzyme group such as amylase, protease, lipase and the like. Generated by the species Koji fungi is dried containing moisture in the range of 2.5% to 3.6% under a state that it has no capability of catalysis.

As described above, the manufacturing method of the present invention could fix various kinds of useful group of enzyme to the base materials by applying a strict control over temperature, humidity and time and cultivating a specified species fungi against the crop without being contaminated with miscellaneous bacteria.

In addition, the present invention has an effect capable of manufacturing the durable nutritious supplemental food of hardly producing any rot due to the fact that the species bacteria and not-required miscellaneous bacteria naturally entered in the food are diminished under a strict continuous control of temperature, humidity and time after ripening process and the group of enzyme useful for the human body are kept still while the capability of catalysis action is kept and the contained moisture in the materials is dried to 4% or less.

The nutritious supplemental food manufactured by this method shows that various composing elements of grain are decomposed into low molecules easily absorbed into the human body or new composing elements are synthesized under an action of enzyme, resulting that a tremendous amount of group of enzyme produced are taken into the human body while they are alive.

With such an arrangement as above, a group of useful enzyme hardly taken by a usual food and some nutritious composing elements easily taken into the human body can be taken when the nutritious supplemental food is taken and at the same time a well-balanced supplementation of nutrition elements and energy can be given to the texture cells to improve environment of cells and activate them, improve metabolism function and immunization function originally held by the human body and further healthy condition can be held. In particular, the present invention is a natural food not provided with any additives and it has an effect not producing any side effects.

### PREFERRED EMBODIMENTS OF THE INVENTION

A preferred embodiment of the present invention will be described as follows.

At first, “unpolished rice” means a black rice (a red rice) cultivated in ancient times and the rice applied in the present invention also includes unpolished usual rice in general. “Embryo” of the unpolished rice occupies about 3% of the entire unpolished rice. The embryo of the unpolished rice contains abundantly minerals or vitamins as well as about 13% of protein, about 17% of lipid and about 9% of ash content or the like.

As the preferred embodiment 1 of the manufacturing method, the mixture substances of embryo (about 37%) and seed coat (about 63%) of selected unpolished rice acting as the major material is put into a wet charger, baked oyster shell powder (particle size #250 to 400 meshes) of about 1% is added and water by about 25 wt % to 35 wt % is absorbed against the entire volume of the major material and bulged out.

The mixed major material bulged with the wet charger (of any sub-base material is also included) is put into a steamer and heated with steam of 100° C. to 120° C. again while its mixing with foreign material is being checked and moisture is being checked for about 45 minutes to 60 minutes.

The mixed major material such as embryo and seed coat completely heated with steam are transferred automatically from the steamer to the cooling device, their heat is released down to about 37° C. to 40° C. or so and cooled. The mixed major materials such as embryo and seed coat heated with steam and just after their cooling are transferred automatically to the mixing device, a small amount of species Koji fungi Aspergills oryzae strain group (about 1/1000 of the amount of base material) is added and they are well mixed to each other.

In this case, the species Koji fungi can be optionally selected from other kinds of species Koji fungi of different characteristics (Aspergills oryzae, Aspergills kawachii, Aspergills sojae, Aspergills awamori, Aspergills usamii) and they can be mixed with the former material in a range of 20% to 30%, respectively. These species Koji fungi is fitted for decomposing sugar and protein.

The species Koji fungi Aspergillis oryzae may produce a plurality of decomposing enzymes such as amylase, protease, lipase and the like to decompose either sugar or protein contained in the base materials into low molecules. The atmosphere suitable for cultivation of the aforesaid species Koji fungi shows a temperature of 35° C. to 37° C. and a humidity of 85% RH to 90% RH and the species Koji fungi may actively secrete some decomposing enzymes under this atmosphere and at this humidity. The species Koji fungi do not work under a humidity of 70% RH or less and almost of it are diminished at a temperature of 42° C. or more.

The mixture weighing about 1 ton, for example, mixed with the aforesaid species Koji fungi is transferred into a closed box type temperature and humidity adjusting machine, its temperature and humidity are adjusted in a stepwise manner within an inner atmosphere temperature of about 31° C. to 37° C. and a humidity range of about 50% RH to 90% RH and the species Koji fungi is cultivated for about 32 to 36 hours.

The temperature and humidity adjusting machine is provided with an opto-electrical optional program setting device, controls a temperature of blown air (also a humidity produced by steam vapor mixed with air) and can make a program over control of a temperature of the mixture during the cultivation of species Koji fungi) under setting of optional numerical values.

In addition, the aforesaid temperature and humidity adjusting mechanism has a culture bed and an agitating device in it in addition to an air conditioner and a humidifier. The agitating device is provided with some scraping vanes and a scraping tool therein.

At the temperature and humidity adjusting machine, the mixture for use in cultivating the species Koji fungi are scraped and agitated by the agitating device and scraped flat.

During a lag phase for promoting growth of the species Koji fungi, the mixed major material may generate heat as the species Koji fungi is cultured and the temperature of the mixed major material at the culture bed is increased automatically and gradually from 5° C. up to 38° C. When the temperature of the mixed major material at the culture bed becomes near 40° C. to 41° C., the mixed major materials at the culture bed are agitated and contacted with fresh air in such a way that action of the species Koji fungi is not deteriorated with heat and the temperature of the mixed major material at the culture bed is forcedly decreased. At this time, the temperature of the mixed major material is dropped to about 32° C. and increased again. This state is continued for a minimum period of 8 hours.

During a growing period promoting ripening of the species Koji fungi, the major material at the culture bed are agitated to adjust its temperature in such a way that the temperature of the mixed major material is increased as the species Koji fungi is cultivated may not exceed 40° C., the atmosphere at the culture bed is kept at 31° C. to 36° C. to cultivate the species Koji fungi. The mixed major material at the culture bed are agitated to cause themselves to be contacted with fresh air and the temperature is forcedly decreased in such a way that when the temperature of the mixed major material becomes near 40° C. to 42° C. after 6 hours, the species Koji fungi may not be deteriorated with over-heating or may not be diminished. After this operation, the temperature is adjusted by agitating the base material at the culture bed in such a way that the temperature of the mixed major material becomes 39° C. to 40° C. and then the species Koji fungi is cultivated for about 11 to 12 hours.

During a rest phase in which the species Koji fungi stops its growth, the humidity is set to be less than humidity 70% RH where the activity of the species Koji fungi is decreased and the atmosphere at the culture bed is increased up to 36 to 37° C. The mixed major material of the culture bed is ripened for 6 hours while the temperature is being adjusted at a range of about 39.5° C. to 40° C. by agitating the mixed major material of the culture bed. At this time, a value of pH of the mixed major material at the culture bed is kept at 6 to 7 or less.

After the mixed major materials have become the ripened species Koji fungi cultivation mixture caused by secreted enzyme of the species Koji fungi, the ripened species Koji fungi cultivation mixture is dried at 45° C. to 53° C. of atmosphere in the culture bed for 8 hours and 30 minutes to 9 hours and 30 minutes, preferably 9 hours and ±10 minutes. The item temperature of the species Koji fungi cultivation mixture at this time is controlled in a range of 44° C. to 46° C. by a method wherein either hot air or moisture at the culture bed is released by agitating the mixed major material at the culture bed and controlling the temperature.

In the case of the atmosphere or the temperature of the species Koji fungi cultivation mixture at this range, the group of enzyme secreted during the cultivation shows that the capability of catalysis action naturally owned by enzyme is not lost and the ripening caused by the group of enzyme is continued, although the action of the species Koji fungi is gradually decreased and the species Koji fungi is diminished by the temperature.

At the time of completion of this drying stage, the ripened base materials have become the dried species Koji fungi cultivation mixture having moisture content of about 2.5% to 3.6%. Incidentally, the contained moisture of normal market distributed rice is about 14% to 15%.

Although under this dried condition, the group of enzyme contained in the dried species Koji fungi cultivation mixture does lose any capability of catalysis action, the group of enzyme is kept still and its action is completely stopped. This group of enzyme accepts moisture within the human body when a person takes food and then the catalysis action is activated.

The dried species Koji fungi cultivation mixture upon completion of the aforesaid stage is applied to a sieve to remove some block solid substances with a size of 5 mm or more and presence of its foreign material is checked. The dry substance is applied to a crushing machine to attain the nutritious supplemental food composed of dried fine powder while a degree of fine powder is being checked.

The fine powder of the aforesaid dried species Koji fungi cultivation mixture is automatically metered, fed into a can, the can is sealed and then the food is sold as a powder-like nutritious supplemental food.

In addition, a mass to be described later is mixed with the fine powder of the species Koji fungi cultivation mixture, the material is formed into granules with a particle size of about 1.2 mm to 1.3 mm by a granulating machine, and they are filled into bags by a partial packaging machine after drying operation with one package being weight of about 3.5 g or 4 g or so to attain a granular product. As required, the powder is formed into tablet by a tablet machine.

A preferred embodiment 2 of a manufacturing method will be described in reference to a case in which Ganoderma lucidum is added as the auxiliary material.

Ganoderma lucidum has been widely used as Chinese medicine since old times due to the fact that it is called that polysaccharide having, as its major substances, various kinds of large amount of glucans contained in the seed activates white corpuscles, causes hormones to be activated and further activates immunity function. Also in the modern medicine, there have been reported in the publications (Chinese Medicine VOL.10, No.6, '86.6) that Ganoderma lucidum has some effects against hyperlipemia syndrome, hepatitis, nervous breakdown, nephritis, diabetes, arrhythmia and other various kinds of diseases.

As composition of Ganoderma lucidum, an analytical data has been reported that it contains rough fiber of 57.6%, carbohydrate of 33.6%, protein of 10.7%, fat of 2.4%, sugar of 1.5% and ash content of 1.0% and the like. However, there is a certain difference in an amount of composition in view of a Ganoderma lucidum-growing district, so that the present inventor mixed three kinds of Ganoderma lucidums produced at three Ganoderma lucidum-growing districts and used them in the embodiment.

As to the size of Ganoderma lucidum, the seed is crushed into a grain size of about 3 mm, and a range of 5 wt % to 15 wt %, in principle 10 wt % of Ganoderma lucidum in respect to an entire amount of Ganoderma lucidum is fed into the aforesaid wet charger together with the embryo or the seed coat described above. The materials are bulged by the wet charger and heated with steam of 100° C. to 120° C. for about 45 to 60 minutes. The staged performed afterwards are similar to those described in the Embodiment 1.

The Embodiment 3 of the manufacturing method will be described in reference to a case that unpolished rice is added as auxiliary material. If the unpolished rice is applied as it is, the major materials having fine grain size positioned below the unpolished rice are pressed by a weight of the unpolished rice to cause some patterns to be produced at the species Koji fungi cultivation, so that a range of 5 wt % to 15 wt %, in principle 10 wt % in respect to an entire amount of it is fed into the aforesaid wet charger together with the embryo or the seed coat described above. The material are bulged by the wet charger and heated with steam of 100° C. to 120° C. for 45 to 60 minutes. The species Koji fungi cultivation stages performed afterwards are similar to those described in the Embodiment 1 of the manufacturing method.

With the foregoing, 80 wt % to 95 wt % of the mixed major material of embryo and seed coat in respect to the entire amount is used. In this case, although the seed coat of about 63% and the embryo of about 37% are applied, only the embryo can be separated and used in order to improve quality.

Although the baked oyster shell powder is selected bone powder, calcium lactate and crab shell and other materials can be used instead of the baked oyster shell powder. In general, although its amount is about 1 wt % or so, an amount of 5 wt % to 10 wt % can be used as required.

When the baked oyster shell powder is heated with steam together with the major materials or auxiliary materials, the hard seed coat of unpolished rice or unpolished rice and a Ganoderma lucidum are softened by alkaline substance and when they are taken as food, they may easily be digested in the human body.

Since a grain size of the embryo or seed coat is smaller as compared with that of rice particles, if the embryo or the seed coat contains moisture and they are closely contacted to each other, a flow of air is deteriorated and this is not preferable for a mean cultivation of the species Koji fungi of aerobe. In addition, it may cause a propagation of non-welcomed anaerobic bacteria. At the culture bed, the baked oyster shell powder may become a clearance forming material between the major materials or auxiliary materials to prevent the major materials of fine grain size or the auxiliary materials from being closely contacted to each other, air is well contacted with the surface of the major materials or the auxiliary materials in uniform manner and it is possible to attain cultivation in which the mean species Koji fungi are provided.

In addition, the baked oyster shell powder uniformly dispersed in the material neutralize and relieve oxidization of the mixed major material during cultivation of species Koji fungi at the culture bed which is apt to be oxidized. In addition, the baked oyster shell powder may act preferably against growing of enzyme, restrict sour of food, make taste of the food tender and provide a preferable influence to odor. Further, the baked oyster shell powder decomposed with acid is easily digested within the human body and is utilized as calcium ion of secondary information transmittance substance.

As the Embodiment 4 of the manufacturing method of the present invention, the mixed major material of seed coat and embryo cultivated with species Koji fungi is of 45 wt % to 75 wt %; the following materials as auxiliary materials;

| | |
|---|---|
| unpolished rice processed | 5 wt % to 12 wt % |
| with species Koji fungi; | |
| Ganoderma lucidum processed | 5 wt % to 12 wt % |
| with species Koji fungi; | |

the following materials as sub-materials; and

| | |
|---|---|
| soybean protein; | 15 wt % to 33 wt % |
| Spirulina; | 3 wt % to 8 wt % |
| liquid glucan; | 3 wt % to 8 wt % and |
| pollen; | 2 wt % to 6 wt % |

They are added and mixed to each other to have a total amount of 100% under the following mixing ratio and optional combination.

The liquid glucan is extracted liquid of polysaccharide extracted from a single cell weed and a concentration of its solid substance is about 0.5 to 1.0%.

Spirulina is one of a family of weed and more than 60% of the weed is protein having a well-balanced composition of amino acid.

Spirulina, liquid glucan, soybean protein and pollen or the like are mixed into the powder before its packaging operation. These materials mainly contain protein, they are decomposed and absorbed into the human body through the group of enzyme taken together with them to produce a mutual nutritious effect.

In addition, as a mass in granulation and tablet granulation, it is possible to add

| | |
|---|---|
| lactose; | 13 wt % to 24 wt % |
| glucose; | 4 wt % to 12 wt % and |
| edible oil and fat | 2 wt % to 6 wt % |

in respect to an entire amount of it.

As edible oil and fat, it is possible to use rape oil and safflower oil (linolic oil). Further, it is possible to blend vitamin C having a powerful reducing action and also having an anti-hardening action of arteries within a range of 50 mg to 300 mg per 20 g of the nutritious supplemental food.

In addition, in the case that grains other than embryo and seed coat are applied as major materials, each of rice, wheat and corn or the like of 10 wt % to 35 wt % and each of millet, and barnyard grass or the like of 5 wt % to 10 wt % are optionally combined and blended to each other, they are heated with steam to cultivate the species Koji fungi.

In this case, each of the embryo and the seed coat portion of the grain can be used in a restricted manner.

As a blending rate of the sub-materials of the species Koji fungi cultivated dry substances of the nutritious supplemental food, they can be mixed to each other within a range of

| | | |
|---|---|---|
| 1) | species Koji fungi embryo, seed coat; | 70 wt % to 90 wt % |
| 2) | species Koji fungi cultivated | 5 wt % to 15 wt % |
| | unpolished rice; and | |
| 3) | species Koji fungi Ganoderma lucidum | 5 wt % to 12 wt % |

In addition, as the sub-materials supplementing the nutrition to each of the dried species Koji fungi cultivation mixture described above,

| | | |
|---|---|---|
| 1) | soybean protein; | 15 wt % to 33 wt % |
| 2) | Spirulina; | 3 wt % to 8 wt % |
| 3) | liquid glucan; | 3 wt % to 8 wt %, and |
| 4) | pollen | 2 wt % to 6 wt % |

or the like can be combined and blended in each of the blending ratios above in respect to a total amount of 100. A result of analysis (accumulation) of the nutritious composition per 100 g of the nutritious supplementing food (the basic item=mixture with unpolished rice of 10 wt % of the present invention manufactured in this way is as follows.

| | | |
|---|---|---|
| 1) | vitamin B1; | 1.6 mg to 1.8 mg |
| 2) | vitamin B6; | 2.3 mg to 2.4 mg |
| 3) | vitamin B12; | 5.4 μg to 5.9 μg |
| 4) | protein; | 13 g to 14 g |
| 5) | lipid; | 18 g to 21 g |
| 6) | moisture; | 1.5 g to 3.6 g |
| 7) | energy; | 375 Kcal to 459 Kcal |
| 8) | acid value; | 36.5 to 50.8 |
| 9) | activation for | 490 unit/g to 770 unit/g |
| | eliminating active oxygen; | |
| 10) | lipase potency; | 540 U/g to 560 U/g |
| 11) | acid protease potency; | 1.90 U/g to 250 U/g |
| 12) | neutral protease potency; | 350 U/g to 430 U/g |
| 13) | caustic protease potency; | 190 U/g to 230 U/g |

Checking this result makes it clear that a large amount of groups of vitamin B essential for activating cells in the physical body are contained. In addition, it can be estimated that the enzyme potency is fulfilled and it is superior in digestion, fat metabolism and protein metabolism.

The present inventor performed the following experiments in order to check the activity for eliminating active oxygen (.O₂— and .OH).

1. Measuring Method and Specimen

Under application of a spin trapping method using an electronic spin resonance, a rate of restriction against a system for generating active oxygen (super oxide anion radical: .O₂—, hydroxy radical: .OH) of the nutritious supplemental food of the present invention was studied. As the measuring device, the present inventor used ESR device (Model: Electron Spin Resonance JES-FR30) manufactured by Nihon Denshi Co., Ltd.

As solvent, phosphoric.acid damper liquid of 0.2 m-mol was used, and

(1) hypoxanthine solution of 2 m-mol (HPX),

(2) diethylene triaminepenta acetic acid (DETAPAC) solution of 5.5 m-mol,

(3) each of these solutions was set to 20 μl, 35 μl and 50 μl, respectively, and then 50 μl of 0.4 U/ml xanthineoxidase solutions (XOD) was added to each of them, after agitating them, 15 μl (9.2 mol) of 5,5-dimethyl-1-pynoline-N-oxide (DMPO) was mixed with O₂— adduct generated within a quartz cell being applied as a trapping agent, thereafter it was measured as a spectrum of DMPO-O₂— generated within an exclusive quartz cell off 120 mm.

A signal intensity of DMPO-O₂— was calculated as a relative intensity against a signal intensity of an internal standard Mn²⁺.

All the sample aqueous solution were adjusted as 0.2 mg/ml, 2 mg/ml and 20 mg/ml and a restriction against production of .O₂— at 50 μl was measured as DMPO-O₂— spectrum. As a control process, phosphoric acid damper liquid was used as solvent and a rate of restriction (%) against it was calculated.

2. Measurement of Activation of Diminishing .OH with ESR

As to an activation for diminishing hydroxy radical, it was measured as a restriction against a Fenton reaction.

That is, each of sample aqueous solution of 50 μl of 0.2 mg/ml, 2 mg/ml and 20 mg/ml was added to 75 μl of DTAPAC=diethylene triaminepenta acetic acid of FeSO₄— of 1 m-mol, 20 μl of DMPO=5,5-dimethyl-1-pynoline-N-oxide and 0.1 m-mol of 75 μl of H₂O₂ were added, after agitation of 2 seconds, it was measured by ESR as DMPO-OH with aforesaid flat cell.

A sweeping was started after 60 seconds after adding H₂0₂. Phosphoric acid damper liquid was similarly used for a control process and it was calculated as a rate of restriction (%) against it.

All the spectrum analysis with ESR device were carried out under the following condition.

Under an output of 4 mW,

| | |
|---|---|
| magnetic field sweeping width: | 335.6 mT |
| magnetic field modulation: | 0.1 mT |
| amplification factor: | 125 |
| sweeping time: | 2 minutes |
| responding time: | 0.1 second |
| measurement temperature: | room temperature |
| Adjustment of measuring specimen | |

As the nutritious supplemental food of the present invention, four kinds of a basic food, food with Spirulina, food with Ganoderma lucidum and food with liquid glucan were selected as measurement species.

Dose of all samples was adjusted to 20 mg/ml and measured.

3. Result of Measurement

Result of Measurement of Activation for Diminishing .02— with ERs (Cumulative Average)

As a result of experiment, average values in reference to the activation for diminishing .02— and to the sample of 20 mg/ml. were attained as follows:

| | | |
|---|---|---|
| (1) | basic food (90% of embryo, seed | 89% |
| | coat and 10% of unpolished rice) | |
| (2) | Basic food (88% of embryo, seed | 81% |
| | coat and 12% of unpolished rice) | |
| (3) | food with 5% of liquid glucan | 78% |
| | (10% of unpolished rice, 30% of | |
| | soybean protein) | |
| (4) | food with 5% of Spirulina | 76% |
| | (10% of unpolished rice, 30% of | |
| | soybean protein) | |
| (5) | food with 10% of Ganoderum lucidum | 72% |
| | (10% of unpolished rice) | |

As indicated in the above result, the food with liquid glucan and the food with Spirulina and the like also showed high activation for diminishing .02—. However, in view of the blending ratio of the materials, the rate of the embryo and the seed coat shows a high value, so that it can be concluded that this high activation for diminishing .02— is caused by the major material such as the embryo and the like. Accordingly, even if the blending ratio of the major materials, the embryo and the seed coat is about 55 wt % or so, it is apparent that the activation for diminishing .02— can keep 70%.

As described above, it has been confirmed that the nutritious supplemental food of the present invention is remarkably superior in activity for eliminating active oxygen within the human body.

This fact has been confirmed that the nutritious supplemental food has a medical effect as found in a Chinese medicine for restricting various kinds of diseases produced from disorders caused by the active oxygen in the human body, in particular, a syndrome of high blood pressure and a syndrome of hyperlipemia and for activating an entire metabolism.

An efficient amount of nutritious supplemental food in which a person (weighing 60 kg) continues to take for keeping healthy condition is 10 g to 25 g per day, preferably 20 g and the food is taken in divided three times.

An experiment concerning an action of restricting auto-immunization type I diabetes using NOD mouse which is a model animal of the autoimmunization type I diabetes mellitus was carried out as follows at the Animal Experimental Center of Tokyo Medical College in order to check a function to restrict occurrence of diabetes provided by the nutritious supplemental food of the present invention.

(1) Test Experimental Animal:

NOD/Crj, female (in this series, usually, a male mouse hardly has symptoms of diabetes)

(2) Applied Food:

The food of the present invention with 10% of Ganoderma lucidum was dissolved in distillation water far a bacteria reducing injection and applied.

(3) Experimental Group:

(a) Contrast Group:

10 mg/g-weight per day of distillation water for a bacteria reducing injection was applied through the mouth with a stomach sonde needle at evenings of Monday through Friday.

(b) Applied Group:

0.33 mg/g-weight per day of food of the present invention was dissolved in distillation water for a bacteria reducing injection and was applied through the mouth with a stomach sonde needle at evenings of Monday through Friday.

As for both group (a) and group (b), x-ray radiated solid fodder was applied as continuous food.

(4) Breeding Density:

Five mice or less/cage:

(a)—Contrast Group:

15 mice, 3 cages

(b)—Applied Group:

15 mice, 3 cages

(5) Experimental Period:

From the fourth week after birth and for 20 continuous weeks.

(6) Test Items During the Experimental Period:

Weight Measurement:

Once/one week—Individual mouse was identified by an ear piercing method and measured every Thursday.

Blood Sugar Measurement:

Once/four weeks—Blood sugar measurement method—Easy blood sugar measuring instrument.

Urine Sugar Test and Confirmation of Symptoms:

From the 13th week, a urine sugar test was performed at a frequency of twice to three times/week.

As to the individual experimental animal, following items were applied.

(a) Blood sugar was directly measured.

(b) Symptoms was confirmed when a blood sugar value was 200 mg/dl or more.

(c) As to individual mouse showing symptoms, some organs such as pancreas, lever and kidney or the like were taken out after all blood (heparin processing) were taken (textural retrieving method).

(d) In the urine sugar test, a urine sugar test paper was used.

(e) Dissection was carried out in reference to a protocol separately made.

Results of experiment performed as described above were as follows.

(a) Accumulative rate of symptoms of the contrast group was 53.3% upon completion of experiment (the 175-th day).

(b) Accumulative rate of symptoms of the applied group was 11.1% even upon completion of experiment (the 175-th day) and an effect of restricting against symptoms was acknowledged in a statistics manner.

(c) As to the rate of symptoms of antoimmunization type I diabetes of the applied group (accumulative), a meaningful restriction against symptoms was acknowledged from the 167-th day as compared with that of the contrast group (refer to Table 1).

[image]

As apparent from the Table 1, the symptoms of the antoimmunization type I diabetes were acknowledged in the contrast group from the 132nd day. The rate of symptoms was remarkably increased from the 156th day. At the 175th day, the rate of symptoms of autoimmunization type I diabetes of about five times was indicated against the applied group.

At the applied group against it, the symptoms of autoimmunization type I diabetes was confirmed after the 145th day. The rate of symptoms ranging from the 152nd day to the 175th day is about 11.1% in stable state. Therefore, it is apparent that the effect of restriction against the symptoms of the autoimmunization type I diabetes appears stably in a positive manner in the applied group.

That is, in contrast to the contrast group, it was clearly acknowledged in the applied group that the applied group has an effect of restriction to about ⅕ of a rate of symptoms of the autoimmunization type I diabetes. Such a remarkable result as described above is not yet confirmed in any type of prior art foods.

The present inventor studied a change in immunization texture of islet of Langerhans of pancreas in NOD mouse to which the nutritious supplemental food of the present invention is applied. Insulin producing cells are dyed into charcoal grey color with insulin antibody, cells other than insulin producing cells are dyed into red with a mixture of three kinds of antibodies (i.e. antiglucagon, antisomatostatin and pancreatic polypeptide) and a result of observation through an electronic microscope was as follows.

Contrast Group:

High degree of langerhans island inflammation was acknowledged in all individual mice. At the islet of Langerhans of individual mouse showing symptoms, a texture image was observed that only the producing cells other than the insulin producing cells are collected and present. At the individual mice not producing any symptoms, the islet of Langerhans of pancreas with only the insulin producing cells other than insulin producing cells and the islet of Langerhans of pancreas with slight amount of insulin producing cells were observed in a substantial similar rate.

Applied Group of the Food of the Present Invention:

Increased production of epithelial cells of a conduit of pancreas was remarkable. Further, at the epithelial cells of the conduit of pancreas, the insulin producing cells and the producing cells other than insulin occurred here and there.

Group of the insulin producing cells that are assumed to be separated and grew from the epithelial cells of the conduit of pancreas were occasionally observed.

Such an increasing in texture was not acknowledged in the contrast group. In addition, although a high degree of langerhans island inflammation was similarly observed in the applied group, the remaining insulin producing cells were apparently large in number as compared with that of the contrast group.

This fact shows that taking of the nutritious supplemental food of the present invention into the physical body has an effect of restriction against inflammation of beta cells of pancreas secreting insulin and recovering in reproducing cells. It can be sufficiently acknowledged that this fact has an effect of restriction against symptoms of diabetes with this food. In the case that an amount of dose against the aforesaid NOD mouse is compared with a weight of human body (60 kg), it corresponds to about 21 g per day.

In view of an increasing number of adult diseases, they are highly related to hyperlipemia of diseases (high cholesterol, high neutral fat) at circulatory organs (high blood pressure syndrome, arteriosclerosis, cerebral infarct, stricture of the heart, and mycocardial infarction), and as their complication, it is possible to indicate high blood sugar, high blood pressure syndrome and chronic hepatitis and the like.

As to the effect of taking nutritious supplemental food against hyperlipemia or the like, the present inventor will indicate an example of clinical data provided by Doctor Satoh, an adviser of the applicant's company, as follows.

### CLINICAL EXAMPLE 1

Adult male (age: 39) with mental uneasiness caused by stress, feeling of fatigue, high degree of disorder in lever function, and diabetes: Blood sugar value at the time of hunger is 309 mg/dl (normal value is 110 mg/dl or less).

Upon continuous taking of the foods of the present invention (one food with 10% of Ganoderma lucidum and the other food with 5% of liquid glucan) by 21 g every day, the blood sugar value at the time of hunger became 118 mg/dl on the 23-th day. Also his diabetes was recovered to a substantial normal state.

### CLINICAL EXAMPLE 2

Adult female (age: 56) with diabetes and hyperlipemia:

She continued to take the food of the present invention (food with 10% of unpolished rice) before her normal meal by 21 g every day. The blood sugar value was decreased from 144 mg/dl to 128 mg/dl within a week. Neutral fat was changed from 500 mg/dl to 263 mg/dl.

### CLINICAL EXAMPLE 3

Adult female (age: 55) with chronic pancreatitis: Although his γ GTP value of lever function was in a range of 250 IU to 450 IU (normal value is 60 IU or less), he continued to take the food of the present invention (food with 10% of Ganoderma lucidum) by 21 g every day, resulting in that γ GTP value of lever function was decreased to 100 IU or less after two months.

### CLINICAL EXAMPLE 4

Adult female (age 72) with a hypertension of about 168 MmHg:

Upon continuous taking of the food of the present invention. (food with 10% of unpolished rice) by 21 g per day for the purpose of keeping health, her blood pressure on the 25-th day was decreased down to a lower value of 108 mmHg and to an upper value of 135 mmHg.

### CLINICAL EXAMPLE 5

Adult female (age: 62) with diabetes and a blood sugar value at the time of hunger 307 mg/dl (normal value is 110 mg/dl or less).

Upon continuous taking of the food of the present invention (food with 5% of liquid glucan) without any internal medicines by 21 g every day, her blood sugar value on the 45-th day became 181 mg/dl.

### CLINICAL EXAMPLE 6

Adult female (age: 56) with diabetes and hyperlipemia: a blood sugar value of 152 mg/dl and a neutral fat value of 464 mg/dl (normal value is about 120 mg/dl).

Upon continuous taking of the food of the present invention (food with 5% of liquid glucan) by 21 g every day, her blood sugar value on the 55th day became 128 mg/dl and her neutral fat value became 263 mg/dl.

### CLINICAL EXAMPLE 7

Adult male (age: 39) with chronic pancreatitis, diabetes, blood sugar value of 170 mg/dl and diabetes index (HbAl) of 8.8 (normal value is 7.7 or less):

Upon continuous taking of the food of the present invention with 10% of unpolished rice and the food with 5% of liquid glucan by 21 g every day, his blood sugar value after 8 weeks became 118 mg/dl and his diabetes index became 7.4.

### CLINICAL EXAMPLE 8

Adult female (age: 71) with hypertension and neutral fat of 280 mg/dl:

Upon continuous taking of the food of the present invention with 5% of liquid glucan by 21 g every day, her neutral fat after 2 weeks became 156 mg/dl.

### CLINICAL EXAMPLE 9

Adult male (age: 65) with diabetes, disorder of lever function, blood sugar value of 337 mg/dl and neutral fat of 287 mg/dl:

Upon continuous taking of one food of the present invention with 5% of liquid glucan and the other food with 5% of Spirulina by 21 g every day, respectively, his blood sugar value became 244 mg/dl and neutral fat value became 190 mg/dl in three weeks.

### CLINICAL EXAMPLE 10

Adult male (age: 68) with diabetes and being treated with injection of insulin and with his blood sugar value of 326 mg/dl:

Upon continuous taking of one food of the present invention with 5% of liquid glucan and the other food with 5% of Spirulina by 21 g every day, respectively, his blood sugar value became 172 mg/dl in 80 days.

Upon checking these clinical examples, it can be acknowledged that a continuous taking of a specified amount of nutritious supplemental food of the present invention causes a fat metabolism to be activated and hyperlipemia is improved. Carbohydrate metabolism is activated and a high blood sugar value is improved.

Further, the present invention is not limited to the aforesaid preferred embodiments and it is possible to supplement nutritious elements by fulfilling vitamin, ginseng powder, turmeric powder, Chinese matrimony vine powder, garlic powder, sea weed (as a source of iodine) or the like, for example, or snapping turtle powder, dried snake powder, dried shells, dried fish or crab shell powder and skim powder milk and the like as animal protein in a can or by adding them just before packaging operation.

Although the nutritious supplemental food of the present invention can be eaten as it is or it may also be applicable that the food is dissolved into liquid such as fruit juice or milk or the like and further mixed with jerry, cream, breads, noodles and devil'is tongue and the like. In addition, at the drying stage, it is also possible that a temperature of the atmosphere is not increased and vacuum is utilized for performing a drying operation.

What is claimed is:

1. A method for manufacturing a nutritious supplemental food comprising:
as its initial step, heating a mixture of embryo and seed coat of unpolished rice as a major material with steam and cultivating a mixed major material having said heated mixture mixed with Aspergills oryzae strain group of species Koji fungi for 32 hours to 36 hours on a culture bed with atmosphere of 31° C. to 37° C. and humidity of 90%RH to 100%RH while both temperature and humidity being adjusted in a stepwise manner;
as its guiding step, cultivating said mixed major material for a minimum period of 8 hours agitating the mixed major material in the culture bed in order to stop an increasing in a heat generating temperature of said mixed major material generated by cultivating the species Koji fungi and thereby dropping its temperature to a scope ranging from 32° C. to 41° C.;
as its growing step, keeping the atmosphere at 31° C. to 36° C., keeping a temperature of said mixed major material in the culture bed at 40° C. to 43° C., continuing cultivation for 6 hours, thereafter cultivating the mixed major material for 11 hours to 12 hours controlling a temperature of the mixed major material in the culture bed at 39° C. to 40° C. and ripening it;
as its stabilizing step, keeping the atmosphere at a temperature of 36° C. to 37° C. and a humidity of 90%RH to 100%RH, adjusting a temperature of said mixed major material in the culture bed to 39.5° C. to 40° C. and ripening it for 6 hours to attain a species Koji fungi cultivation mixture;
as a drying step, holding the ripened species Koji fungi cultivation mixture in the culture bed at 44° C. to 46° C. for 8 hours and 30 minutes to 9 hours and 30 minutes without controlling an increasing of heat generating temperature of it, thereafter killing the species Koji fungi contained in the species Koji fungi cultivation mixture on the culture bed with hot heat in which a temperature of said species Koji fungi cultivation mixture is controlled to 39° C. to 40° C., to attain a dried species Koji fungi cultivation mixture with moisture content of 2.5% to 3.6% without loosing any capability of catalysis of each enzyme group contained in said species Koji fungi cultivation mixture with heat of said species Koji fungi cultivation mixture; and
under a combination of these steps, mixing said dried species Koji fungi cultivation mixture applied as a major substance with a sub-material which does not cultivate any species Koji fungi.

2. A method for manufacturing a nutritious supplemental food according to claim 1, wherein full-particle of the unpolished rice is crushed into half-sized particle, said crushed unpolished rice of 10 wt % to 15 wt % of a total amount of food as an auxiliary material is mixed with a mixture of embryo and seed coat of the unpolished rice as a major material and they are heated with steam.

3. A method for manufacturing a nutritious supplemental food according to claim 1, wherein seed of a Ganoderma lucidum as an auxiliary material is crushed, said crushed seed of 10 wt % to 12 wt % of a total amount of food is mixed with a mixture of embryo and seed coat of the unpolished rice as a major material and they are heated with steam.

4. A method for manufacturing a nutritious supplemental food according to claim 1, wherein baked oyster shell powders with grain size of #250 mesh to #400 mesh are mixed with said mixture as a major material and heated with steam.

5. A nutritious supplemental food wherein:
a group of Aspergills oryzae strain of species Koji fungi is mixed in a mixture of embryo and seed coat of unpolished rice acting as a major material, cultivated and ripened to generate the species Koji fungi cultivation mixture kept under a state in which it includes groups of enzyme such as amylase, protease, lipase and the like generated by Koji fungi;
Koji fungi in said species Koji fungi cultivation mixture are made dead under a state in which capability of catalysis of said groups of enzyme is not lost; and
said species Koji fungi cultivation mixture is formed into dried material with included moisture being in a range of 2.5% to 3.6%, said dried species Koji fungi cultivation mixture is applied as a major substance and a sub-material of powder form is mixed with said dried mixture.

6. A nutritious supplemental food according to claim 5, wherein said dried species Koji fungi cultivation mixture is formed into powder, mixed with a mass, and changed into particles with a size of diameter of 1.2 mm˜1.3 mm.

7. A nutritious supplemental food according to claim 5, wherein unpolished rice powder having species Koji fungi cultivated is mixed at a rate of 5 wt % to 15 wt % of a total amount of food in said dried species Koji fungi cultivation mixture.

8. A nutritious supplemental food according to claim 5, wherein in said dried species Koji fungi cultivation mixture Ganoderma lucidum powder of 5 wt % to 12 wt % of a total amount of food is mixed in advance and cultivated with species Koji fungi.

9. A nutritious supplemental food according to claim 5, wherein soybean protein acting as sub-material of 15 wt % to 33 wt % of a total amount of food are added to said dried species Koji fungi cultivation mixture.

10. A nutritious supplemental food according to claim 5, wherein Spirulina acting as sub-material of 3 wt % to 8 wt % of a total amount of food are added to said dried species Koji fungi cultivation mixture.

11. A nutritious supplemental food according to claim 5, wherein 50 mg to 300 mg of vitamin C are added to 20 g of the powder of said dried species Koji fungi cultivation mixture.
